(19) United States
(12) Patent Application Publication
(10) Pub. No.: US 2024/0100274 A1
(43) Pub. Date: Mar. 28, 2024

(54) DEVICE FOR MONITORING WEARING OF A MASK

(71) Applicant: Jack Joseph Trama, Gilbert, AZ (US)

(72) Inventor: Jack Joseph Trama, Gilbert, AZ (US)

(21) Appl. No.: 18/465,968

(22) Filed: Sep. 12, 2023

Related U.S. Application Data
(60) Provisional application No. 63/409,067, filed on Sep. 22, 2022.

(51) Int. Cl. A61M 16/00; A61M 16/06

(52) U.S. Cl. CPC A61M 16/0003; A61M 16/0683; A61M 2205/13; A61M 2205/18; A61M 2205/3306; A61M 2205/3327; A61M 2205/3569; A61M 2205/44; A61M 2205/582; A61M 2205/583; A61M 2205/587; A61M 2205/8206

(57) ABSTRACT

According to an aspect of the present invention, there is provided a system for detecting the removal of mask from the face of a user comprising: a sensor housing configured to attach to a mask via one or more attachment mechanisms; one or more sensors in the sensor housing configured to detect a change in position of a mask on the face of a user; a processor configured to analyze sensor data to determine whether a change in position detected by the one or more sensors exceeds a predefined threshold; and an alerting device for activating an alert upon the predefined threshold change in position being exceeded.

### CROSS-REFERENCE

This application claims priority to U.S. Provisional Patent Application 63/409,067, filed Sep. 22, 2022.

### BACKGROUND

Embodiments of the invention described in this specification relate generally to monitoring wearing of medical masks, including continuous positive airway pressure (CPAP) masks and systems, and more particularly, to a stand-alone mask removal audible beeper device that attaches to a CPAP mask worn by a person for breathing conditions, and activates when the mask is unknowingly removed from the face of the person.

Many people suffering from Obstructive Sleep Apnea (OSA) unknowingly remove their CPAP mask from their face while sleeping. A person does not know when they have removed the mask while asleep. Sleeping without the mask can cause serious and sometimes fatal health problems.

Prior art lacks a stand-alone device for the purpose of detecting when a mask such as an oxygen mask or a CPAP mask is removed (inadvertently, unknowingly, etc.) and alerting the person to put the mask back on.

In fact, even many CPAP machines do not have an audible beeper system built into their devices. This is because CPAP machine must stop the flow of air, and in doing so the machine turns off immediately when the mask is removed from the person's face. No audible sound is heard for the user to know their mask has been removed. Many newer CPAP machines do not have a beeper installed in them at all and, therefore, a person would not be informed when they have removed the mask while asleep. Sleeping without the mask can cause serious and sometimes fatal health problems.

Therefore, what is needed is an audible beeper device that attaches to a mask to audibly alert a user when the mask is removed and provide them an opportunity to put the mask back on their face to resume treated sleep.

### SUMMARY OF INVENTION

According to an aspect of the present invention, there is provided a system for detecting the removal of mask from the face of a user comprising: a sensor housing configured to attach to a mask via one or more attachment mechanisms; one or more sensors in the sensor housing configured to detect a change in position of a mask on the face of a user; a processor configured to analyze sensor data to determine whether a change in position detected by the one or more sensors exceeds a predefined threshold; and an alerting device for activating an alert upon the predefined threshold change in position being exceeded.

### BRIEF DESCRIPTION OF THE DRAWINGS

FIG. 1 conceptually illustrates a side elevation view of a person wearing a CPAP mask with a mask removal audible beeper device attached to the CPAP mask in some embodiments.

FIG. 2 conceptually illustrates a perspective view of a stand-alone mask removal audible beeper device in some embodiments.

FIG. 3 conceptually illustrates a front elevation view of the stand-alone mask removal audible beeper device in some embodiments.

FIG. 4 conceptually illustrates a rear elevation view of the stand-alone mask removal audible beeper device in some embodiments.

FIG. 5 conceptually illustrates a side elevation view of the mask removal audible beeper device in some embodiments.

FIG. 6 conceptually illustrates another side elevation view of the mask removal audible beeper device in some embodiments while the mask removal audible beeper device sounds an audible alarm in connection with a distance-based infrared sensor of the mask removal audible beeper device detecting that the mask is removed.

### DETAILED DESCRIPTION

In the following detailed description of the invention, numerous details, examples, and embodiments of the invention are described. However, it will be clear and apparent to one skilled in the art that the invention is not limited to the embodiments set forth and that the invention can be adapted for any of several applications.

Some embodiments provide a stand-alone mask removal audible beeper device that attaches to a CPAP mask, worn by a person for sleep apnea and related breathing conditions, and activates when the mask is unknowingly removed from the face of the person. In some embodiments, the stand-alone mask removal audible beeper device attached to the CPAP mask is battery-operated. In some embodiments, the stand-alone mask removal audible beeper device attached to the CPAP mask uses the battery, a microcontroller, and an infrared based distance sensor to detect when the mask is removed and warn the user of the problem.

As stated above, many people suffering from Sleep Apnea unknowingly remove their CPAP mask from their face while sleeping. A person does not know when they have removed the mask while asleep. Sleeping without the mask can cause serious and sometimes fatal health problems.

Currently, there is no stand-alone device that exists on the market for the purpose of detecting when the mask is removed (inadvertently, unknowingly, etc.) and alerting the person for the reason of putting the mask back.

In fact, even many CPAP machines do not have an audible beeper system built into their devices. This is because CPAP machine must stop the flow of air, and in doing so the machine turns off immediately when the mask is removed from the person's face. No audible sound is heard for the user to know their mask has been removed. Many newer CPAP machines do not have a beeper installed in them at all and, therefore, a person would not be informed when they have removed the mask while asleep. Sleeping without the mask can cause serious and sometimes fatal health problems. Embodiments of the stand-alone mask removal audible beeper device attached to the CPAP mask described in this specification solve such problems by way of a mask removal audible beeper device that attaches to any CPAP mask and alerts the user with a series of audible beeps once the mask is unknowingly removed from their face. When the device senses the mask was removed, an audible alarm triggers awakening the user so they can put on the mask back on their face.

Embodiments of the stand-alone mask removal audible beeper device attached to the CPAP mask described in this specification differ from and improve upon currently existing options. In particular, some embodiments differ by utilizing a mask removal audible beeper device which can be attached to whatever CPAP mask a user wears. The audible beeper will alert them immediately once their mask is removed and provide them an opportunity to put the masks back on their face to resume treated sleep. This provides a vast improvement over the existing options, of which no stand-alone device exists for this purpose.

In addition, the fewer and fewer existing, older CPAP machines with audible beepers installed are not typically activated by the manufacturer by default because when a user's CPAP mask is unknowingly removed, the CPAP machine must stop the flow of air, thus endangering the user, and in doing so the machine itself turns off immediately. Accordingly, these CPAP machines do not function to alert the user of the mask coming off, as no audible sound is heard (the machine turns off) for the user to know their mask has been removed. Also, many newer CPAP machines do not have a beeper installed at all. Thus, the existing CPAP machines typically lack an audible beeper system built into their devices.

The stand-alone mask removal audible beeper device attached to the CPAP mask of the present disclosure may be comprised of the following elements. This list of possible constituent elements is intended to be exemplary only and it is not intended that this list be used to limit the stand-alone mask removal audible beeper device attached to the CPAP mask of the present application to just these elements. Persons having ordinary skill in the art relevant to the present disclosure may understand there to be equivalent elements that may be substituted within the present disclosure without changing the essential function or operation of the stand-alone mask removal audible beeper device attached to the CPAP mask.
1. BATTERY RETAINER COIN 20MM SMD (“Battery Retainer”)
2. CAP CER 0402 0.1UF 16V X7R 10% (“Ceramic Capacitor” or “0.1 uF X7R 10% 16 VDC Multilayer Ceramic Capacitor”)
3. CAP CER 4.7UF 25V X5R 0603 (“Ceramic Capacitor” or “4.7 uF 25 VDC Multilayer Ceramic Capacitor”)
4. CAP CER 10UF 16V X6S 0805 (“Ceramic Capacitor” or “10.0 uF 16 VDC X6S Multilayer Ceramic Capacitor”)
5. CAP CER 1UF 10V X7S 0402 (“Ceramic Capacitor” or “1 uF 10 VDC
X7S Multilayer Ceramic Capacitor”)
6. CAP CER 0402 10NF 50V X7R 20% (“Ceramic Capacitor” or “10 nF X7R 20% 10 VDC Multilayer Ceramic Capacitor”)
7. LED RED CLEAR CHIP SMD (“Red Indicator LED”)
8. LED GREEN CLEAR CHIP SMD (“Green Indicator LED”) 9.
16 mm, 25 V, 70 dB, Surface Mount (SMT), Piezo Audio Transducer Buzzer (“Audible Output Device”)
10. RES 47K OHM 1% 1/16 W 0402 (“Resistor”)
11. RES SMD 1K OHM 5% 1/16 W 0402 (“Resistor”)
12. RES 10K OHM 0.1% 1/10 W 0402 (“Resistor”)
13. RES 470K OHM 1% 1/16 W 0402 (“Resistor”)
14. SWITCH SLIDE SPDT 300MA 6V (“On/Off Toggle Switch”)
15. SWITCH TACTILE SPST-NO 0.02A 15V (“Tactile Switch”)
16. STM32L432KCU6 (“Micro-controller” as the main control unit “MCU”)
17. SENSOR OPT AMBIENT 12SMD VL6180 (“Distance-Based Infrared Sensor” or “Proximity/Ambient Light Detection Sensor” or “Infrared (IR) Sensor”)
18. Micropower, 150 mA Low-Noise Ultra Low-Dropout CMOS Voltage Regulator, 5-pin SOT-23 (“Voltage Regulator” or “Linear Regulator”)
19. CR2032 Battery (“Battery”)
20. Components 1-19, above, included on a Printed Circuit Board (“PCB”)
21. Plastic Housing (“Housing” or “Exterior Housing”)
22. Silicone Strap (“Strap”)

The various elements of the stand-alone mask removal audible beeper device attached to the CPAP mask of the present disclosure as presented in the figures and in this Specification may be related in the following exemplary fashion. It is not intended to limit the scope or nature of the relationships between the various elements and the following examples are presented as illustrative examples only. The mask removal audible beeper device comprises a circuit board (PCB) encased in a plastic housing. All the components 1-19 are part of the PCB which performs all the functions of the mask removal audible beeper device. The stand-alone mask removal audible beeper device is powered using a standard CR2032 lithium coin cell battery or other suitable battery. The stand-alone mask removal audible beeper device is optimized for low power consumption. The stand-alone mask removal audible beeper device consists of a power source, a micro-controller (MCU), and an infrared based distance sensor. The micro-controller may be an STM32L4 micro-controller, but any other suitable micro-controller or control unit would work. The MCU is connected to a proximity/ambient light detection sensor (VL6180 or other compatible proximity sensor) through an I2C interface. The power source is a battery (CR2032 or other compatible battery) that supplies 3 volts. The battery voltage is stepped down to 2.8 volts using a linear regulator to power the proximity/ambient light detection sensor. For the user interface, the stand-alone mask removal audible beeper device has a tactile switch and two indicator LEDs. There is also a power switch that allows for the stand-alone mask removal audible beeper device to be fully turned off. A piezoelectric buzzer provides audible output. The stand-alone mask removal audible beeper device is designed to be in sleep mode when powered on. The tactile button wakes up the micro-controller. If the button is pressed for a long duration, it will activate the proximity/ambient light detection sensor and set it to measure distance at a frequency of 2 Hz. At that time, the micro-controller goes to sleep. The proximity/ambient light detection sensor performs periodic distance measurements and if the distance increases beyond a threshold amount, it will trigger the micro-controller to sound the audible alarm. The audible alarm is triggered by a 4 kHz 3-volt square wave at 50% duty cycle. This allows for maximum sound output by the piezo buzzer. When the audible alarm goes off, the stand-alone mask removal audible beeper device can be turned off using the button. If left on to buzz, it will turn off after a specified amount of time. The system also tests the battery voltage every time it is turned on and if it is lower than a certain threshold, it will turn on the red LED indicator to indicate low battery. The top cover of the housing gets push-fitted on top of the bottom housing piece to encase the PCB circuit board assembly and encapsulate the associated components completely. The exterior housing also has holes or cut-outs for the indicator LEDs and a feature designed for the button.

The stand-alone mask removal audible beeper device attached to the CPAP mask of the present disclosure generally works by way of the circuit board (PCB) encased in the exterior housing. All the components are part of the circuit board and perform the requisite functions for operation of the stand-alone mask removal audible beeper device. The exterior housing has two parts—the top and the bottom. The bottom part is what the circuit board (PCB) and its components, as well as the silicone strap, mounts to. The silicone strap allows the mask removal audible beeper device to be strapped to the CPAP mask. The top cover gets push fitted onto the bottom of the housing to entirely encapsulate the PCB and its components.

To make the stand-alone mask removal audible beeper device attached to the CPAP mask of the present disclosure, the device would be assembled from several parts including, without limitation, a circuit board (PCB) encased in a housing (e.g., a plastic housing), a micro controller, an audible device (that is, a piezo audio transducer buzzer), a battery for power, and other components as noted above. The main components of the PCB include the power source, the micro-controller (MCU), the proximity/ambient light detection sensor (IR sensor) to detect distance, and the piezoelectric buzzer to provide the audible alert when the mask is detected to be removed. Specifically, the IR sensor performs periodic distance measurements and if the distance increases beyond the threshold distance, it will trigger the micro-controller to sound the piezoelectric buzzer. The alarm (piezoelectric buzzer) is triggered by a 4 kHz 3-volt square wave at 50% duty cycle. This allows for maximum sound output by the piezoelectric buzzer.

To use the stand-alone mask removal audible beeper device attached to the CPAP mask of the present disclosure, a person would simply attach the fully assembled mask removal audible beeper device to their CPAP mask by way of the silicon strap and at a location that suits their needs. For example, the mask removal audible beeper device is attached to a CPAP mask strap in several of the figures. Once attached to the CPAP mask, the user would put on the CPAP mask, power up/turn on the mask removal audible beeper device and go to sleep. If the CPAP mask is inadvertently and unknowingly removed while sleeping, the sensor will detect the distance, and the audible alarm of the mask removal audible beeper device will be triggered to sound an audible alarm that will awaken the user.

Additionally, the stand-alone mask removal audible beeper device attached to the CPAP mask of the present disclosure can be adapted for wireless communication by adding Bluetooth capability to communicate with a mobile app and a smart watch, a smart band, or other smart device or wearable, which are commonly worn by, affixed to, or nearby the user's body. For instance, communicating with the smart watch or band will allow the watch or band to vibrate and awaken a user through vibration. Also, the user will be able to use the audible beeper alert in conjunction with vibration, or just use one alert without the other. For example, the vibration option will be useful for those with bed partners.

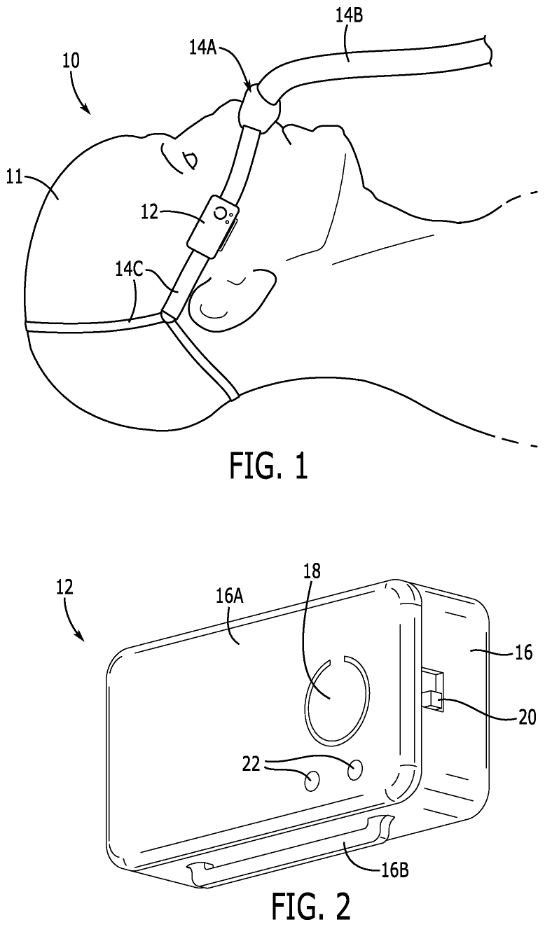

FIG. 1 conceptually illustrates a side elevation view of a person wearing a CPAP mask with a mask removal audible beeper device attached to the CPAP mask in some embodiments.

Element 10 is the forehead of the wearer.

Element 11 is the skull of the wearer.

Element 12 is a sensor housing according to an embodiment of the present invention.

Element 14A is a nasal pillow.

Element 14B is hose connecting to a medical machine.

Element 14C is a strap connecting a medical mask to the head of a wearer.

FIG. 2 conceptually illustrates a perspective view of a stand-alone mask removal audible beeper device in some embodiments.

Element 12 is a sensor housing according to an embodiment of the present invention.

Element 16 is a side of the sensor housing.

Element 16A is another side of the sensor housing.

Element 16B is an attachment point for a harness connecting an embodiment to a medical mask.

Element 18 is an activation button for arming and disarming a device according to an embodiment.

Element 20 is an activation button for arming and disarming a device according to an embodiment.

Element 22 is a battery life indicator.

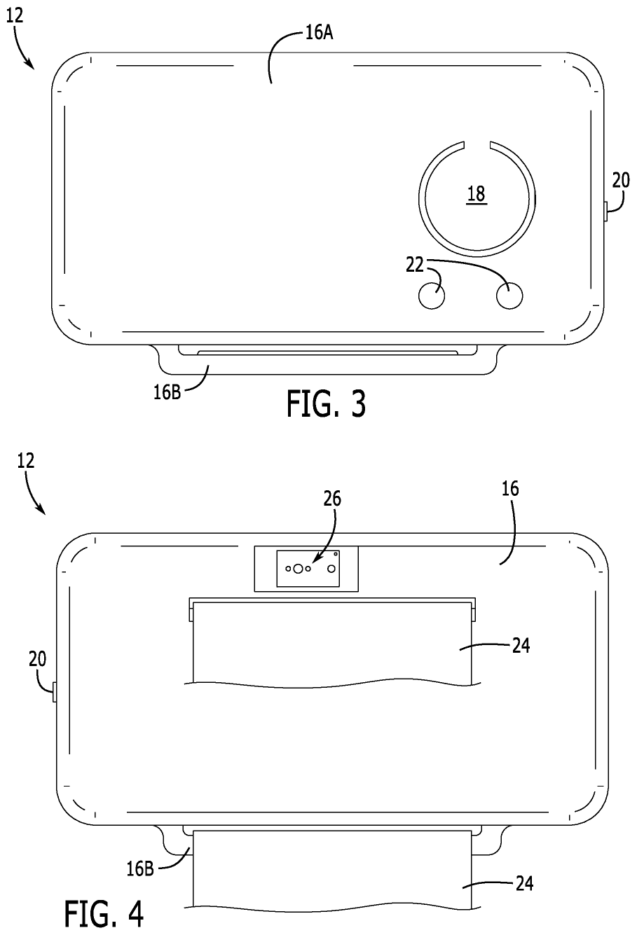

FIG. 3 conceptually illustrates a front elevation view of the stand-alone mask removal audible beeper device in some embodiments.

Element 12 is a sensor housing according to an embodiment of the present invention.

Element 16A is another side of the sensor housing.

Element 16B is an attachment point for a harness connecting an embodiment to a medical mask.

Element 18 is an activation button for arming and disarming a device according to an embodiment.

Element 20 is an activation button for arming and disarming a device according to an embodiment.

Element 22 is a battery life indicator.

FIG. 4 conceptually illustrates a rear elevation view of the stand-alone mask removal audible beeper device in some embodiments.

Element 12 is a sensor housing according to an embodiment of the present invention.

Element 16 is a side of the sensor housing.

Element 16B is an attachment point for a harness connecting an embodiment to a medical mask.

Element 20 is an activation button for arming and disarming a device according to an embodiment.

Element 24 is a strap connecting the device according to an embodiment with a medical mask.

Element 26 is a sensor beam sensing the position of the head of the wearer.

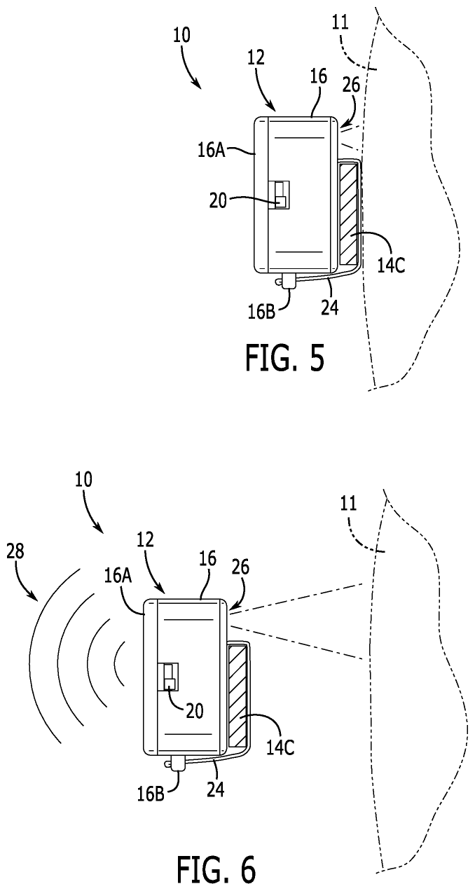

FIG. 5 conceptually illustrates a side elevation view of the mask removal audible beeper device in some embodiments.

Element 10 is the forehead of the wearer.

Element 11 is the skull of the wearer.

Element 12 is a sensor housing according to an embodiment of the present invention.

Element 14C is a strap connecting a medical mask to the head of a wearer.

Element 16 is a side of the sensor housing.

Element 16A is another side of the sensor housing.

Element 16B is an attachment point for a harness connecting an embodiment to a medical mask.

Element 20 is an activation button for arming and disarming a device according to an embodiment.

Element 24 is a strap connecting the device according to an embodiment with a medical mask.

Element 26 is a sensor beam sensing the position of the head of the wearer.

FIG. 6 conceptually illustrates another side elevation view of the mask removal audible beeper device in some embodiments while the mask removal audible beeper device sounds an audible alarm in connection with a distance-based infrared sensor of the mask removal audible beeper device detecting that the mask is removed.

Element 10 is the forehead of the wearer.

Element 11 is the skull of the wearer.

Element 12 is a sensor housing according to an embodiment of the present invention.

Element 14C is a strap connecting a medical mask to the head of a wearer.

Element 16 is a side of the sensor housing.

Element 16A is another side of the sensor housing.

Element 16B is an attachment point for a harness connecting an embodiment to a medical mask.

Element 20 is an activation button for arming and disarming a device according to an embodiment.

Element 24 is a strap connecting the device according to an embodiment with a medical mask.

Element 26 is a sensor beam sensing the position of the head of the wearer.

Element 28 is an illustration of sound waves of an audible alert.

Having thus described a few particular embodiments of the invention, various alterations, modifications, and improvements will readily occur to those skilled in the art. Such alterations, modifications, and improvements are intended to be part of this description though not expressly stated herein, and are intended to be within the spirit and scope of the invention. Accordingly, the foregoing description is by way of example only of several of many potential embodiments, and not limiting.

What is claimed is:

1. A system for detecting the removal of mask from the face of a user comprising:
a sensor housing configured to attach to a mask via one or more attachment mechanisms;
one or more sensors in the sensor housing configured to detect a change in position of a mask on the face of a user;
a processor configured to analyze sensor data to determine whether a change in position detected by the one or more sensors exceeds a predefined threshold; and
an alerting device for activating an alert upon the predefined threshold change in position being exceeded.

2. The system of claim 1, wherein the attachment mechanism comprises one or more of an adjustable clip(s) that can attach securely to the mask strap or frame to hold the sensor, a custom 3D printed mask fitting(s) to accommodate different mask models and sizes, and a harness that wraps around the mask with multiple attachment points for stability.

3. The system of claim 1, wherein the one or more sensors comprise accelerometers, gyroscopes, capacitive touch, or optical sensors to detect changes in orientation, electrical proximity to skin, movement, or light levels indicating mask displacement.

4. The system of claim 1, further comprising a mobile application configured to receive sensor data from the sensor module and indicate an audible, visual or tactile alert.

5. The system of claim 1, further comprising one or more of a vibration motor configured to provide haptic feedback alert when mask removal is detected.

6. The system of claim 1, further comprising a wireless communication module configured to transmit sensor data and receive alert activation signals

7. The system of claim 4, wherein the mobile application is further configured to collect and display aggregated data on mask removal events to provide users with feedback on unconscious behaviors.

8. The system of claim 1, wherein the processor is further configured to detect when a user has repositioned their mask after an alert and track duration of proper positioning.

9. The system of claim 6, further comprising a bedside unit for wirelessly connecting with the sensor module to relay sensor data and alerts indirectly to a mobile device.

10. The system of claim 1, further comprising a rechargeable battery.

11. The system of claim 10, wherein the processor automatically turns the system off to conserve battery based on predefined criteria.

12. The system of claim 1, further comprising a speaker in the sensor module configured to output an audible alert when mask displacement is detected.

13. The system of claim 12, wherein the speaker outputs progressively louder audio alarms until predefined criteria associated with a user response to the alert are met.

14. The system of claim 4, wherein the system connects to a smartphone app via Bluetooth for alerting, tracking and reviewing data.

15. The system of claim 1, wherein the threshold change in position is measured from one or more of the face of the user, a baseline reference position of the sensor and a piece of equipment.

16. A method of monitoring the usage of a medical mask, comprising:
attaching a sensor housing to a mask worn by a user by an attachment mechanism;
measuring, by one or more sensors in the sensor housing, a change in position above a threshold associated with the medical mask being displaced from a proper worn position; and
activating an alert indicator to notify the user to adjust the mask position when mask displacement above the threshold is detected.

17. The method of claim 16, wherein the medical mask comprises a CPAP mask, oxygen mask, protective mask, or cannula.

18. The method of claim 16, wherein detecting displacement comprises detecting changes in orientation, movement, light levels, or electrical proximity to the skin.

19. A sensor device attachable to a mask to detect displacement comprising:
a housing containing one or more sensor(s), one or more processor(s), one or more wireless communications device(s), and an alarm indicator; and
an attachment mechanisms for securing the housing to a medical mask, wherein:
the one or more sensors comprise sensor(s) for detecting changes in orientation, light, movement or electrical conductivity indicating mask displacement;
the one or more processors comprise processor(s) for analyzing sensor data and activating the alarm indicator upon detecting displacement events; and
the one or more wireless devices comprise device(s) with wireless connectivity for transmitting sensor data and receiving control signals.

20. The device of claim 23, wherein the alarm indicator comprises a vibration motor, speaker, lights, or text/voice messages.
